(19) United States
(12) Patent Application Publication
(10) Pub. No.: US 2021/0401532 A1
(43) Pub. Date: Dec. 30, 2021

(54) NEGATIVE PRESSURE PROTECTIVE EQUIPMENT

(71) Applicant: LevMarc LLC, Milford, CT (US)

(72) Inventors: Steven Brian Levine, Weston, CT (US); Andrew Marcus, Westport, CT (US)

(73) Assignee: LevMarc LLC, Milford, CT (US)

(21) Appl. No.: 17/356,813

(22) Filed: Jun. 24, 2021

Related U.S. Application Data
(60) Provisional application No. 63/043,978, filed on Jun. 25, 2020.

(51) Int. Cl. A61B 90/00; A61G 10/00; A61G 10/02

(52) U.S. Cl. CPC A61B 90/05; A61G 10/023; A61G 10/005

(57) ABSTRACT

A protective equipment includes a rotatable window and an extended member secured to a peripheral edge of the rotatable window. The rotatable window may be configured as a shield to be disposed between a patient and a healthcare worker to minimize exposure of the healthcare worker to at least one of the patient's bodily fluids, bacteria, and viruses. A port provides entrance by one or more instruments through the rotatable window or the extended member, thereby providing access to the patient while protecting the healthcare worker from the patient's bodily fluids, bacteria and/or viruses. The protective equipment may include an air flow system configured to receive and filter air expelled by the patient, to pull the expelled air away from the patient's face and to filter/clean the expelled air.

### CROSS-REFERENCE TO RELATED APPLICATIONS

This application claims the benefit of U.S. Provisional Application No. 63/043,978 filed Jun. 25, 2020, which is incorporated herein in its entirety.

### BACKGROUND OF THE DISCLOSURE

Healthcare workers/providers typically wear various protective gear when caring for patients with certain diseases and viral infections, in order to reduce the healthcare workers' potential risks of contracting such diseases and viral infections. Core practices currently implemented include the healthcare worker's use of protective eyewear, and a mask or face shield to protect the mucous membranes of their eyes, nose, and mouth during any procedures and activities likely to generate any splashes or sprays of any secretions, bodily fluids, blood, and the like from patients being attended to.

Face shields, in particular, are commonly used by healthcare workers who take care of novel coronavirus (COVID-19) patients and those with potential COVID-19 infections. COVID-19 is a highly infectious virus that is transmitted through aerosols and may be transmitted through droplets generated when an infected person coughs, sneezes, or speaks. Because a significant reservoir of the novel coronavirus responsible for COVID-19 is within the nasal cavity, nasopharynx, oropharynx, and larynx, specialists who perform procedures on these anatomic regions may have significant work-related exposure to such viral particles. For example, specialists in Otolaryngology-Head & Neck Surgery (Ear, Nose & Throat or ENT) and Dental Medicine may have significant exposure risks. Patients having such procedures performed, other members of staff, and patients visiting such medical offices are also at risk of exposure.

In 2020, the risk of infection transmission of COVID-19 shut down routine patient care, especially office-based procedures such as those common in dentistry. Furthermore, other non-airway procedures of the face, head, and neck may be of concern with respect to aerosols, such as seen in otolaryngology, ophthalmology, oral surgery, neurosurgery, and plastic surgery. This may arise due to a significant reservoir of the novel coronavirus responsible for COVID-19 being within the nasal cavity, nasopharynx, oropharynx, and larynx and/oruse of high-speed power drills and electrocautery, which typically have the greatest association with generating aerosols.

FIG. 1 illustrates a conventional face shield 10. The face shield 10 includes a solid plastic sheath 20 that extends from or is attached to a securing mechanism 30. The securing mechanism 30 is attached to an upper portion of the sheath 20 and secures the face shield 10 to the healthcare worker's head. In order to provide additional protection to the healthcare worker, the healthcare worker may also wear a face mask 40 to cover his/her mouth and nose. For example, a conventional face mask, such as a surgical mask, may be worn under the face shield 10.

FIG. 2 illustrates another version of the face shield 10. The face shield 10 includes the solid plastic sheath 20 positioned around the wearer's face. In FIG. 2, the securing mechanism 30 extends from sides of the solid plastic sheath and is wrapped around the healthcare worker's head.

While healthcare workers wear various protective gear to reduce their risk of exposure, the patient may be left exposed to any diseases and viral infections that can be transmitted from the healthcare workers to the patient. In addition, should the protective gear being worn by the healthcare worker fail, there may be no additional measures for protecting the patient from any airborne viruses and/or bacteria that may be in the room. Similarly, airborne viruses and/or bacteria may be transmitted from the patient to the healthcare worker if no barrier exists to prevent viruses or bacteria from being transmitted from the patient to the healthcare worker.

It has been recommended that some medical offices be configured as Airborne Infection Isolation Rooms (AIIR) (also referred to as negative pressure rooms or areas) for the performance of medical procedures. AIIR serve to trap any potential harmful airborne viruses and/or bacteria and prevent those airborne viruses and/or bacteria from leaving the space of the AIIR, thereby isolating anyone outside the room from exposure to the patient's airborne viruses and/or bacteria. Such AIIR are expensive to build and retrofit to medical buildings and facilities. In addition, they can be cost prohibitive for mid-size and small medical practices.

Accordingly, there is a need for a system that addresses the issues outlined above. There is a further need for a system that can be used for common surgical airway procedures, for example as in otolaryngology (such as tonsillectomy). There is a need for a protective equipment that can be utilized in a medical office space and/or operating room theatre to protect the patient and/or healthcare workers from diseases and viral infections. There is a further need for a protective equipment that provides a personalized negative pressure protective equipment for a patient. There is a need for a cost-effective way to provide negative pressure protection during healthcare procedures. While these needs have been described for exemplary purposes with respect to COVID-19, the needs may relate more broadly with respect to medical procedures and/or infectious diseases.

### BRIEF DESCRIPTION OF THE EXEMPLARY EMBODIMENTS

According to an aspect, the exemplary embodiments include a protective equipment. The protective equipment includes a rotatable window configured as a shield to be disposed between the patient and a healthcare worker to minimize exposure of the healthcare worker to at least one of the patient's bodily fluids, bacteria, and viruses. An extended member is secured to a peripheral edge of the rotatable window and has an inner surface configured to face the patient and an outer surface configured to be opposite the patient (e.g. to face away from the patient, or disposed opposite the inner surface, with. Thickness of the extended member therebetween). Some embodiments may have a port. For example, the extended member may include a port that extends between the inner surface and the outer surface. The port may be configured to provide entrance by one or more instruments through the extended member, thereby providing access to the patient while protecting the healthcare worker from at least one of the patient's bodily fluids, bacteria and viruses. It is contemplated that the extended member may be particularly suited for substantially inhibiting or trapping micro aerosolized particles from traversing the extended member.

Further embodiments of the disclosure are associated with a protective equipment including a rotatable window, an air flow system configured to pull air expelled by the patient away from the patient's face and filter the expelled air, and an extended member secured to the rotatable window. The rotatable window and the extended member may be configured substantially as described hereinabove. According to an aspect, the extended member is configured for providing access to a patient to minimize exposure of a healthcare worker to at least one of the patient's bodily fluids, bacteria and viruses. The port may facilitate entrance of an instrument through the extended member, thereby providing access to the patient while protecting the healthcare worker from the patient's bodily fluids, bacteria and/or viruses.

Further embodiments of the disclosure are associated with a protective equipment including a rotatable window, an extended member secured to the rotatable window, and a sleeve in communication with an environment internal to the extended member. The rotatable window and the extended member may be configured substantially as described hereinabove. According to an aspect, the extended member is configured for providing access to a patient to minimize exposure of a healthcare worker to at least one of the patient's bodily fluids, bacteria and viruses. The port may facilitate entrance of the sleeve and an instrument through the extended member, thereby providing access to the patient while protecting the healthcare worker from at least one of the patient's bodily fluids, bacteria and viruses. According to an aspect, the protective equipment further includes an airflow system. The air flow system is configured to pull air expelled by the patient away from the patient's face and filter the expelled air.

### BRIEF DESCRIPTION OF THE DRAWINGS

A more particular description will be rendered by reference to exemplary embodiments that are illustrated in the accompanying figures. Understanding that these drawings depict exemplary embodiments and do not limit the scope of this disclosure, the exemplary embodiments will be described and explained with additional specificity and detail through the use of the accompanying drawings in which:

FIG. 1 is a perspective view of a face shield being worn by a healthcare provider, according to the prior art;

FIG. 2 is a perspective view of a face shield configured to be worn by a healthcare provider, according to the prior art;

FIG. 3 is a perspective view of a negative pressure protective equipment, including a rotatable window and an extended member having a port, according to an embodiment;

FIG. 4A is a side view of a negative pressure protective equipment, illustrating a rotatable window in a pivoted configuration, according to an aspect;

FIG. 4B is a side view of the negative pressure protective equipment of FIG. 4A, illustrating the rotatable window prior to being pivoted;

FIG. 5 is a side, perspective view of a ceiling-mounted negative pressure protective equipment, according to an embodiment;

FIG. 6 is a top view of a negative pressure protective equipment, according to an embodiment;

FIG. 7A is a top view of a barrier configured for use with a negative pressure protective equipment, according to an embodiment;

FIG. 7B is a cross-sectional view of the barrier of FIG. 7A, illustrating upper and lower barriers, according to an embodiment;

FIG. 8 is a top view of a sleeve configured for use with a negative pressure protective equipment, according to an embodiment;

FIG. 9 is a perspective view of a portion of an air flow system configured for use with a negative pressure protective equipment, according to an embodiment; and

FIG. 10 is a top view of a negative pressure protective equipment having an airflow system integrated with a rotatable window, according to an embodiment.

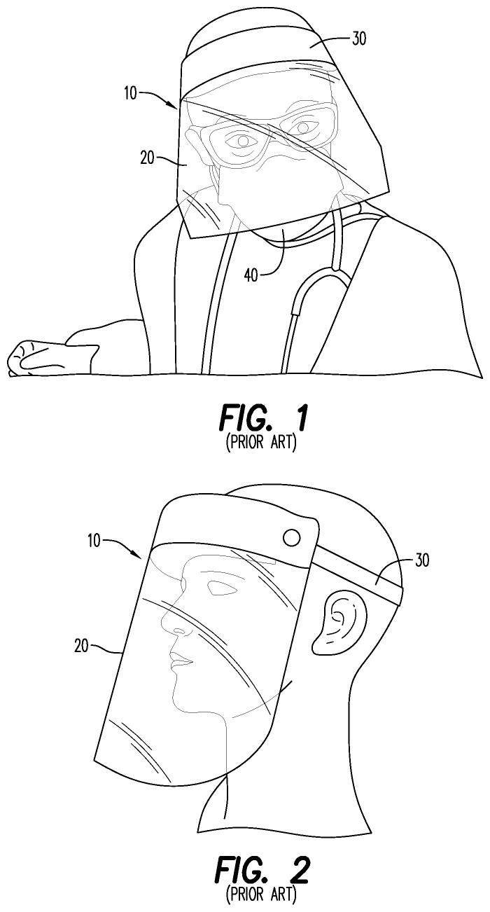

Various features, aspects, and advantages of the exemplary embodiments will become more apparent from the following detailed description, along with the accompanying drawings in which like numerals represent like components throughout the figures and detailed description. The various described features are not necessarily drawn to scale in the drawings, but are drawn to emphasize specific features relevant to some embodiments.

The headings used herein are for organizational purposes only and are not meant to limit the scope of the disclosure or the claims. To facilitate understanding, reference numerals have been used, where possible, to designate like elements common to the figures.

### DETAILED DESCRIPTION

Reference will now be made in detail to various embodiments. Each exemplary embodiment is provided by way of explanation and is not meant as a limitation and does not constitute a definition of all possible embodiments.

Embodiments described herein relate generally to protective equipment that is particularly suited for patients undergoing medical procedures related to the ear, nose, and oral cavities (including the throat). To be sure, the protective equipment described herein may also be used by persons who are not undergoing medical procedures, such as persons seeking additional protection against the transmission or contraction of various viral and bacterial infections, such as airborne viral and bacterial infections.

For purposes of illustrating features of the embodiments, exemplary embodiments are introduced and referenced throughout the disclosure. These embodiments are illustrative and not limiting and are provided for illustrating the exemplary features of a protective equipment as described throughout this disclosure.

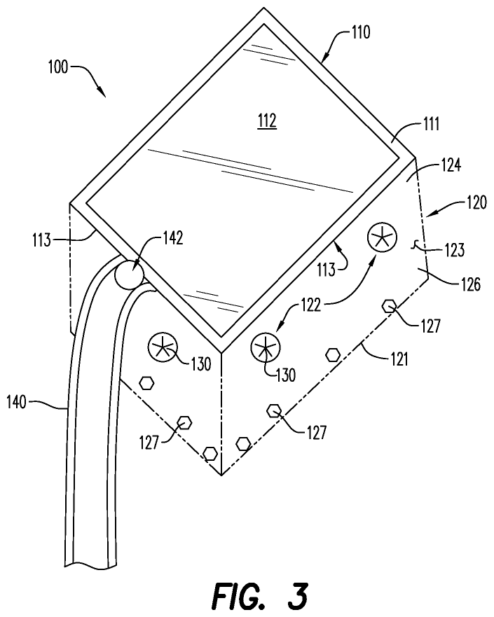

FIGS. 3-4B, FIG. 6 and FIG. 10 each illustrate a protective equipment/negative pressure protective equipment 100. The protective equipment 100 includes a rotatable window 110. The rotatable window 110 may be configured as a shield to be disposed between a patient and a healthcare worker to minimize exposure of the healthcare worker to at least one of the patient's bodily fluids, bacteria, and viruses. The rotatable window 110 may be configured to be elevated so that it is above the patient, and a health care provider can easily view the upper body, including the head, shoulder, and neck areas of the patient, through the rotatable window110. For example, the rotatable window 110 may be configured to be positioned/interposed between a patient (who may be lying down) and a healthcare provider (who may be standing over and/or looking down at the patient).

The rotatable window 110 includes a see-through body 112. The see-through body 112 of the rotatable window 110 may be fully transparent, partially transparent, or partially opaque. According to an aspect, the rotatable window 110 includes at least a portion that is transparent, such as the area around the patient's eyes, ears, or mouth.

The rotatable window 110 may be composed of a number of materials. The see-through body 112 can include at least one of a non-reflective material, a fully transparent material, and an anti-fog material. According to an aspect, the anti-fog material can include a coating of a polyacrylic acid compound, polyvinyl alcohol, and acetylacetone, and optionally sodium silicate.

It is contemplated that the see-through body 112 comprises plastic or glass. According to an aspect, the see-through body 112 can include a thermoplastic film, a thermoset film, or a fibrous material. The see-through body 112 may include polyester. According to an aspect, the rotatable window 110 can include at least one of polyethylene terephthalate (PET), polystyrene-co-polyacrylonitrile, polyolefins, such as polyethylene or their copolymers, polycarbonate, polyacrylates, such as polymethyl methacrylate, polyethyl acrylate, or their copolymers, polysulfone, polyvinylidene chloride or polyvinylidene fluoride, and cellulosic materials such as cellulose acetate butyrate.

According to an aspect, the see-through body 112 is secured in a frame 111. Embodiments of the frame 111 are illustrated in FIGS. 3-4B and FIG. 10. The frame 111 may be composed of any type of rigid material, such as, plastics or metals. According to an aspect, the frame 111 may be pivotably connected to a support arm 140 via a joint (such as a ball joint 142) at a first end of the support arm 140. The ball joint 142 may provide an articulated connection that facilitates three-dimensional movement of the support arm 140. The ball joint 142 may be secured to the support arm 140 by one of more fasteners, such as screws. According to an aspect, the ball joint can include a ball stud, a bearing, a housing, and a retainer. The ball joint 142 may help to facilitate movement (e.g. pivot and rotation) of the rotatable window 110. The ball joint 142 is configured to orient the frame 111 and the see-through body 112 secured therein, such that the rotatable window 110 is vertically and laterally adjustable. For example, the rotatable window 110 may have several degrees of rotational freedom. In some embodiments, the rotatable window 110 may be vertically rotatable/pivotable to an angle between −60 degrees and +60 degrees, and laterally rotatable/pivotable to an angle between −30 degrees and +30 degrees.

In some embodiments, the support arm 140 may be secured or otherwise mounted to a support structure, such as the floor, at a second end of the support arm 140, such that the protective equipment is a floor-mounted protective equipment. The support arm 140 may extend upwardly from a lower or ground surface towards the frame 111 such that the rotatable window 110 is elevated with respect to the body portion of the patient (e.g. when the patient is lying down). In other embodiments, the second end of the support arm 140 may be mounted to a wall, a ceiling, or some other support structure.

According to an aspect, the second end of the support arm may be secured to a mobile structure to facilitate movement of the protective equipment 100 between different locations, such as different rooms or buildings. The mobile structure (not shown) may include wheels, such that the protective equipment 100 can easily be moved between different locations. According to an aspect, the wheels may be configured to collapse or lock so that the protective equipment 100 is stationary when the protective equipment is at its desired location.

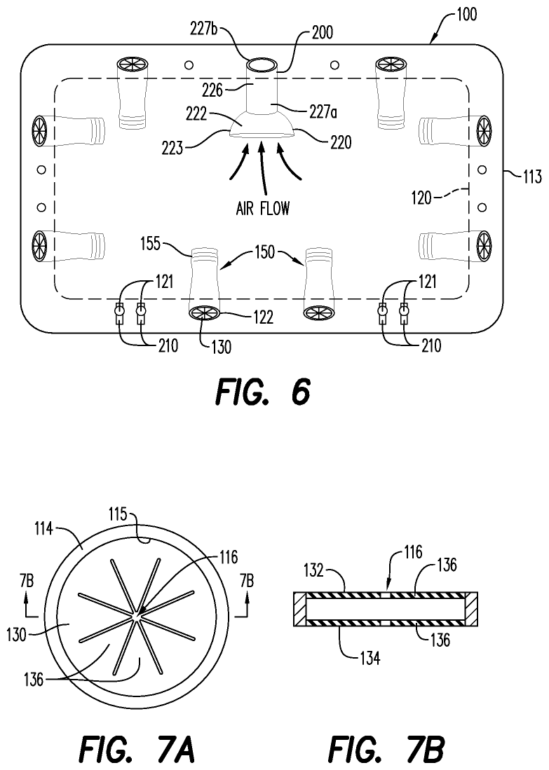

According to an aspect and as illustrated in FIG. 3 and FIG. 6, the protective equipment 100 further includes an extended member/drape 120, which may be secured to a peripheral edge 113 of the rotatable window 110. For example, the extended member 120 may be configured to hang down from the rotatable window 110. The extended member 120 may include a flexible material configured to substantially inhibit or trap aerosolized particles/micro aerosolized particles from traversing the extended member 120. The extended member 120 may including a coating to help inhibit the traversal of aerosolized particles from an interior of the extended member 120 to an exterior of the extended member 120.

The extended member 120 includes a first end 124 and a second end 126 spaced apart from the first end 124. The first end 124 may be removably secured to the peripheral edge 113 of the rotatable window 110. According to an aspect, the first end 124 may be permanently secured to the peripheral edge 113 of the rotatable window 110. The first end 124 may be secured or otherwise connected to the peripheral edge 113 in such a manner as to minimize the release of micro-aerosolized particles when the patient coughs, sneezes, or exhales, or to inhibit the contamination of the patient by micro aerosolized particles when the healthcare provider coughs, sneezes, or exhales. It is contemplated that the extended member 120 may inhibit up to about 95% of micro aerosolized particles from traversing the extended member 120.

The extended member 120 may either drape on top of or around the patient's body in order to cut off or minimize air flow (that is, the exchange of air between an exterior and an interior of the extended member 120) so that it substantially goes through an air flow system 200 (FIG. 6), as described in further detail hereinbelow. As illustrated in FIG. 3, the second end 126 of the extended member 120 may include a securing mechanism 127. The securing mechanism 127 may be configured to secure the extended member 120 in a spaced apart configuration from a body portion of the patient, for example to define a space surrounding the body portion of the patient. The securing mechanism 127 may include at least one of a pressure sensitive adhesive or weights. When the patient lays on an examining or operation table, for example, the weights or the adhesive may secure the extended member to the examining or operation table. In some embodiments, the securing mechanism may comprise multiple components disposed around the second end 126.

According to an aspect, the extended member 120 can be composed of a disposable surgical material. The extended member 120 may be made, at least in part, from fluid repellent or impervious fabrics, to prevent liquid penetration. According to an aspect, the extended member 120 may include a thermally sensitive laminate barrier material composed of thermoplastic polymers. The barrier material may include thermoplastic polymer spunbond fabrics, thermoplastic polymer meltblown fabrics, and various combinations of such spunbond and meltblown fabrics. According to an aspect, the extended member 120 can include one or more thin, breathable films which are resistant to penetration by liquids and pathogens while also providing satisfactory levels of breathability and/or moisture vapor transmission. The film may include a thermoplastic polyolefin, such as, polyethylene, polypropylene, and combinations thereof. According to an aspect, the extended member 120 inhibits the traversal of aerosolized particles from an interior of the extended member 120 to an exterior of the extended member 120.

As illustrated in FIG. 3 and FIG. 6 and according to an aspect, the extended member 120 may further include a port 122 formed therein. The port 122 extends between an inner surface 121 and an outer surface 123 of the extended member 120. The positioning of the port 122 facilitates entrance by one or more instruments through the extended member 120, thereby providing access to the patient while protecting the healthcare worker from the patient's bodily fluids, bacteria, and/or viruses. In other words, the port 122 helps to facilitate access to the patient's orifices during medical procedures being performed by a health care provider. According to an aspect, the port 122 may include a one-way valve that ensures that air may enter inside the extended member 120 but may not exit from it. According to an aspect, multiple ports 122 (FIG. 3 and FIG. 6) may be provided so that multiple health care workers/ providers can simultaneously have access to the patient during procedures or during routine examinations and/or so that easy access is provided to multiple portions of the patient. While the port/ports 122 may facilitate a healthcare provider's access to the patient, it is also contemplated that the port/ports 122 may serve as a path by which gases, such as oxygen and nitrous oxide, can be supplied to the patient.

It is contemplated that at least a portion of the port 122 may be dimensioned so that any desired object may be received therethrough. For example, medical instruments and/or a hand of a healthcare provider may fit through the port 122. The port 122 may include a plurality of ports 122, each port 122 of the plurality of ports 122 being configured substantially as described hereinabove. In this configuration, each port 122 may be spaced apart from another port 122 by a distance that allows the healthcare provider to examiner or treat the patient. In order to facilitate access to the nose or oral cavity, for example, the extended member 120 may include rows of ports 122 that accommodate various instruments or various healthcare providers being able to examine or treat the patient. For example, multiple ports 122 may be provided in one or more sides of the extended member 120.

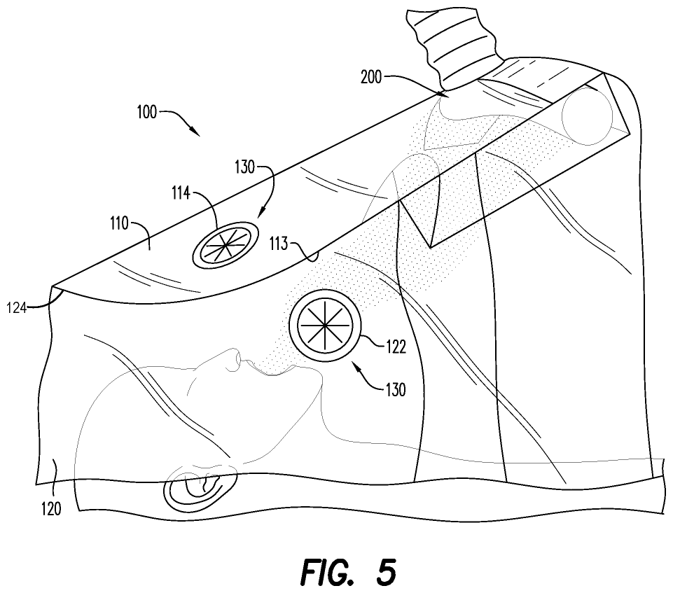

FIGS. 3 and 5 illustrate the protective equipment 100 including a barrier 130 positioned in the port 122. The barrier 130 is shown in more detail in FIG. 7A and FIG. 7B. The barrier 130 includes a periphery/peripheral edge 115 and a plurality of deformable protrusions/bristles 136 extending away from the periphery 115 towards a centralized location 116 of the port 122. The centralized location 116 of the port 122 may form an opening having an inner diameter of up to about 0.5 centimeters (cm). According to an aspect, the barrier 130 helps to secure or guide instruments and the healthcare provider's hand/(s) as they pass through the ports 122 of the extended member 120. According to an aspect, the deformable protrusions 136 of the barrier 130 bend and conform to the instrument or the healthcare provider's hand/(s) as the port 122 is being accessed.

The barrier 130 may include a polymeric material or a copolymer. According to an aspect, the polymeric material can include rubber, silicone, or polypropylene. As would be understood by one of ordinary skill in the art, the barrier 130 may be composed of any material that is sufficiently deformable to provide access to the patient's facial orifices when an instrument is positioned therethrough, but sufficiently stiff so that it closes the gap between the instrument and the periphery 115 of the port 122. For example, in some embodiments the barrier 130 may be configured to minimize the size of the opening of the port 122, while allowing effective passage of instruments and/or the healthcare provider's hand through the port 122 (e.g. closing off unused portions of the port 122 opening).

According to an aspect, the plurality of protrusions 136 comprises multiple flexible layers. As illustrated in FIG. 7B, the barrier 130 may include an upper barrier 132 and a lower barrier 134, with each of the upper barrier and the lower barrier including the plurality of deformable protrusions 136. The protrusions 136 of one barrier may vary in size, shape, material, and quantity from the protrusions 136 of other barriers 130. According to an aspect, for barriers 130 having upper barriers 132 and lower barriers 134, the protrusions 136 may be aligned. Alternatively, the upper barrier 132 and the lower barrier 134 may vary in orientation, so that the protrusions 136 of the upper barrier 132 and the lower barrier 134 are misaligned and/or are diametrically opposite to each other (not shown).

The negative pressure protective equipment 100 may further include an air flow system 200. The air flow system 200 can be configured to pull air expelled by the patient away from the patient's face and filter/clean the expelled air. The air flow system 200 may generate an amount of negative pressure that initiates air flow. It is contemplated that the level of negative pressure may be set according to patient comfort. The air flow system 200 may be used with an awake patient, who may desire a particular negative pressure level. According to an aspect, the air flow system 200 may be used with a patient under general anesthesia.

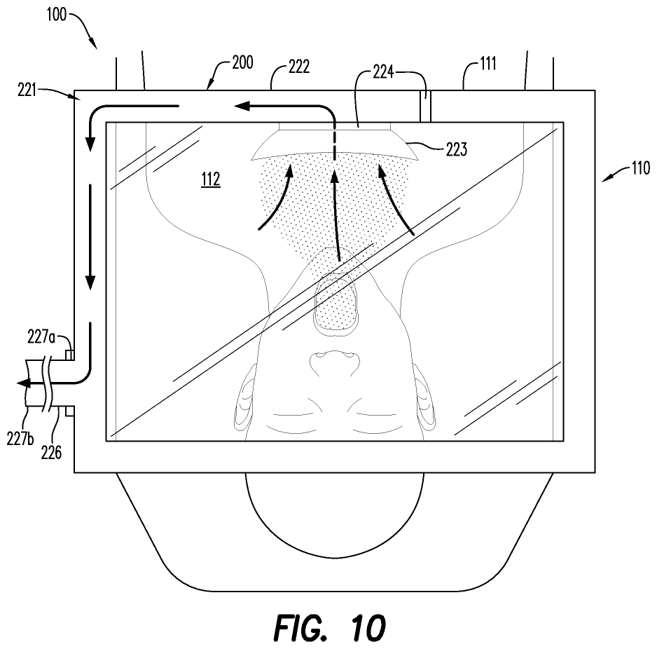

An exemplary air flow system 200 is illustrated in FIG. 6 and FIG. 10. The air flow system 200 is in communication with an internal environment of the negative pressure protective equipment 100 formed by the arrangement of the rotatable window 110 and the extended member 120. This internal environment will essentially include any air being breathed out by the patient and potential droplets associated with the patient's breath, the patient's saliva, and any fluids that may be associated with the patient's maxillofacial areas and the patient's ears. As shown in FIG. 6, the air flow system 200 includes an air valve/intake valve 210 and an air outlet 220 spaced apart from the intake valve 210. The air outlet 220 may further be connected to air filtration system (not shown).

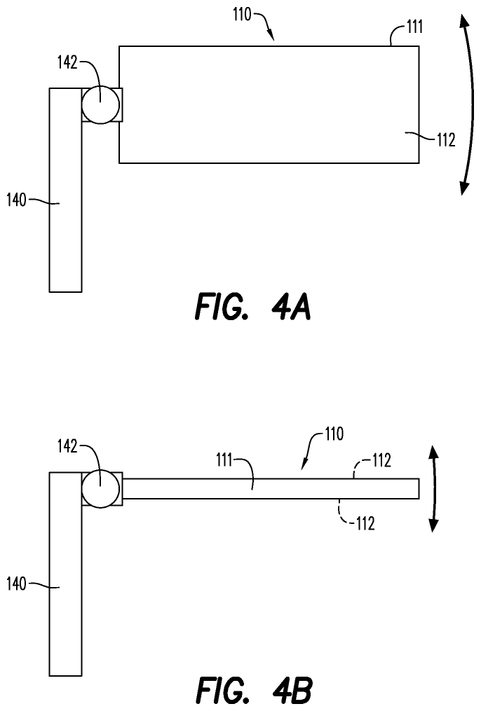

The intake valve 210 may be positioned adjacent a portion of the rotatable window 110 that would be closest to the patient, such as adjacent the support arm 140 illustrated in FIG. 4A and FIG. 4B. The intake valve 210 may be movable between open and closed positions. The intake valve 210 may also be configured to receive and filter air from the environment external to the face shield 110. According to an aspect, the intake valve 210 is a passive valve that receives air from an environment external to the negative pressure protective equipment 100. In this configuration, the intake valve 210 only operates when air is pulled through it—this can occur when the patient breathes in or when air is pulled by a vacuum by an air outlet mechanism (described in further detail hereinbelow). A user, such as the patient, may take normal breaths, without having air being forced into the internal environment of the negative pressure protective equipment 100. The intake valve 210 may be equipped with one or more filters (FIG. 6) configured to filter the air from the environment external to the rotatable window 110 prior to the filtered air being received into the internal environment.

The air outlet 220 may be spaced apart from the air intake 210, and may be configured to pull, via a vacuum-type suction or pull, the air expelled by the patient away from the patient's face and filter/clean the expelled air. When the patient exhales, the exhaled/expelled air typically only travels to the air outlet 220 where it can be filtered. The expelled air may be transported from the internal environment to an external environment (for example, the room or area where the patient is located, such as a medical treatment room, ENT's office or dental office) so that the expelled air can be filtered. According to an aspect, the expelled air is transported, via the air outlet 220, to an air filtration system.

The intake valve 210 and the air outlet 220 work together such that when a patient breathes in, the patient has access to air from the external environment, and when a patient breathes out, the vacuum-configured air outlet 220 pulls the expired air so the expired air can be filtered. The path of the patient's inhaled air and exhaled air, when wearing the negative pressure protective equipment 100, may function similar to the flow path of air in a negative pressure room. As would be understood by one of ordinary skill in the art, the air flow system 200 facilitates the entrance of air into the internal environment created by virtue of the arrangement of the rotatable window 110 and the extended member 120 so that the patient can inhale the air, then pulls the patient's exhaled air out of the internal environment. After the expired air is filtered by one or more filters 224 (FIGS. 9-10), the filtered expired air may be released back into the external environment.

As illustrated in FIG. 6 and FIG. 9, the air outlet 220 may include an air suction conduit 222. The air suction conduit 222 may include a hose 226. The hose 226 may include a plastic or metal material. According to an aspect, the air suction conduit 222 may include a National Sanitation Foundation (NSF)/American National Standard Institute (ANSI) Certified material. For example, and without limitation, one or more parts or components of the air suction conduit 222, such as the hose 226, may be formed from at least one of polyvinyl chloride (PVC), chlorinated polyvinyl chloride (CPVC), polyethylene (PE), polyethylene of raised temperature (PE-RT), cross-linked polyethylene (PEX), acrylonitrile-butadiene-styrene (ABS), polyphenylsulfone (PPSU), nylon, polyurethane (PUR), polystyrene (PS), polyphenylene ether (PPE), acetal or polyacetal (POM), polyethylene terephthalate (PET) and foam polyvinyl chloride (FPVC), brass, copper and rubber. The hose 226 includes a first end 227a and a second end 227b. One or more second filters 224 may be disposed within the hose 226 at one of the first end 227a, the second end 227b, and any position between the first and second ends 227a, 227b. According to an aspect, the first end 227a of the hose 226 is connected to an intake section/a wide intake 223 in communication with the internal environment of the negative pressure protective equipment 100. The intake section 223 has an outer diameter, at its largest cross-section, that is wider than an outer diameter of the hose 226.

In some embodiments, the second end 227b of the hose 226 may be connected to (e.g. in fluid communication with) the air valve 210 (not shown). According to an aspect, the second end 227b of the hose 226 may be connected to the air filtration system. The air filtration system may include a High Efficiency Particulate Air (HEPA) filter, Ultraviolet (UV) light treated filter, Ultraviolet Germicidal Irradiation (UVGI), and/or Ultra-Low Particulate Air (ULPA), such that the air filtration system filters air particles, bacteria and viruses exhaled by the patient (i.e., the patient's exhaled/expelled air). Other filtration systems, although not explicitly outlined herein, are contemplated by this disclosure. The air filtration system may be portable so that it can be transported from one location to another location.

FIG. 10 illustrates an alternate embodiment of the air flow system 200 having the air suction conduit 222 extending through the frame 111 of the rotatable window 110. In this configuration, the frame 111 may include a flow chamber 221 that provides a path for the flow of the patient's expelled air towards the external environment or towards the air filtration system. The first end 227a of the hose 226 is in fluid communication with the flow chamber 221, and the second end 227b of the hose is connected to the air filtration system (not shown).

As described hereinabove and illustrated in FIG. 3 and FIG. 6, the extended member 120 includes a port 122. When a healthcare provider accesses the patient's orifices through the port 122, any airborne particles or liquids will be pulled by the air suction conduit 222 towards the second filters 224 and/or towards the air filtration system. While multiple ports 122 are illustrated in FIG. 5 and FIG. 6, it is contemplated that the extended member 120 may include a single port 122 (FIG. 5) at a location that provides access to the orifice that the healthcare provider would like to examine, treat, or operate on. As illustrated in FIG. 5, for example, it is further contemplated that the rotatable window 110 may include a port 114, within which a barrier 130 is secured. In some embodiments, the port 114 may be similar to the port 122.

FIG. 5 illustrates an alternate embodiment of the protective equipment 100, whereby the protective equipment is ceiling-mounted, rather than floor mounted. The protective equipment of FIG. 5 includes a rotatable window 110, an extended member 120, and an air flow system, each of which are configured substantially as described hereinabove. According to an aspect, the rotatable window 100 illustrated in the embodiment of FIG. 5 is a frameless rotatable window. In this configuration, the first end 124 of the extended member 120 is removably secured to the peripheral edge 113 of the rotatable window 110. The air flow system 200 may be mounted to a ceiling, so that it is suspended or otherwise elevated with respect to a table (for medical examination and procedures, such as treatment or operating procedures). The distance between an outlet of the air flow system 200 and the table or a patient on the table, may be adjustable by one or more mechanisms, as would be understood by one of ordinary skill in the art. In any event, the air flow system is configured to pull air expelled by the patient away from the patient's face and filter/clean the expelled air. The protective equipment 100 in FIG. 5 may be substantially stationary, due to the ceiling-mounted nature of the protective equipment 100 illustrated in FIG. 5. In some embodiments, the floor mounted protective equipment illustrated in FIG. 3, for example, may provide ease and flexibility of installation and may be moveable between different locations.

Some embodiments of the disclosure may further be associated with a protective equipment 100 including a rotatable window 110, an extended member 120 and a sleeve 150 extending through a port 122 formed in the extended member 120. The rotatable window 110 and the extended member 120 may be configured substantially as described hereinabove and illustrated in FIGS. 3-5, for example. Thus, for purposes of convenience and not limitation, the various features and aspects of the rotatable window 110 and the extended member 120 are not repeated hereinbelow.

FIG. 6 illustrates an embodiment of the protective equipment 100 including the sleeve 150. The sleeve 150 may be formed as an extension of the extended member 120 or may be separately connected to the extended member 120. The sleeve 150 may extend around the port 122 formed in the extended member 120.

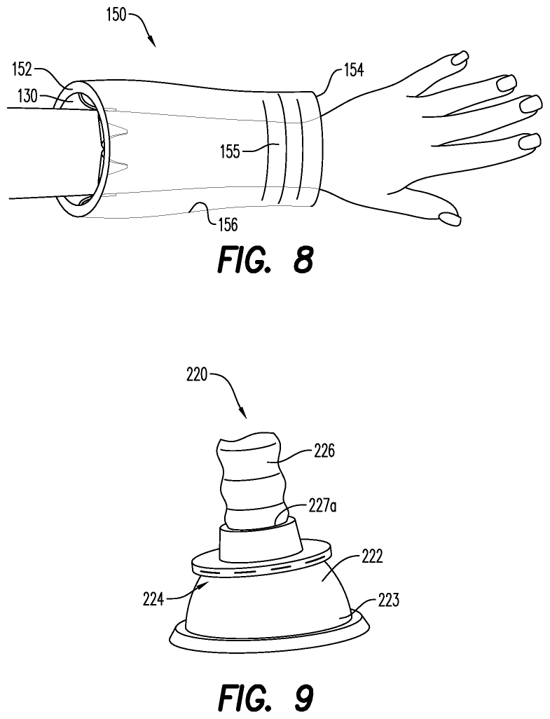

FIG. 8 illustrates in detail an exemplary sleeve 150 as shown in FIG. 6. For purpose of illustration clarity, the sleeve 150 is shown independently. As illustrated in FIG. 8, the sleeve 150 includes a first end/first open end 152 and a second end/second open end 154 spaced apart from the first end 152. A passage 156 extends between the first end 152 and the second end 154 and is configured for receiving an arm of the healthcare provider so that the healthcare provider can assess or treat the patient through the extended member. According to an aspect, the sleeve 150 has a length of about 6 inches, although in various embodiments the length of the sleeve 150 may be more or less. The sleeve 150 is configured to extend under the rotatable window 110, so that a healthcare worker an easily monitor the movement of their hands and any instruments being utilized.

The first end 152 of the sleeve 150 may include a barrier 130 positioned in the passage 156 or in the port 122. The barrier 130 may be configure substantially as described hereinabove and illustrated in FIG. 7A and 7B. Thus for purposes of convenience and not limitation, the various features of the barrier 130 are not repeated hereinbelow. The barrier 130 may be configured to bend and conform to the healthcare worker/operator's arm, and is flexible enough to bend as the arm passes through, but also stiff enough so that it closes the gap between the arm and the border of the first open end 152. According to an aspect, the second end 154 of the sleeve can include an elastic portion 155 configured to securely wrap around the healthcare worker's wrist. A healthcare provider may use gloves that are receivable within the passage 156 of the sleeve and the elastic portion may fasten around a periphery of the gloves to further secure the gloves to the healthcare provider's hands.

It is contemplated that one or more of the components of the protective equipment 100 (for example, the rotatable window and/or the extended member) may be suitable for one-time/single use, so that they are discarded after patient use. Alternatively, one or more of the components of the protective equipment 100 may be suitable for appropriate sterilization and disinfecting procedures after use, which may allow reuse.

It is contemplated that one or more portions of the protective equipment 100 may include a fibrous material/fabric. The fibrous material may include a fibrous batt, such as a nonwoven batt, a nonwoven fabric, and other fibrous structures or composites, which generally include individual fibers or threads which are interlaid, but not in an identifiable manner as in a knitted fabric. In an embodiment, the fibrous material is needle punched to entangle the various fibers and provide Z-directional strength. As a result of the needle punching process, at least some fibers may extend substantially in the Z direction of the fabric may be mechanically entangled with each other. In some instances, the entangled fibers are generally wrapped around and/or intertwined with other fibers of the fabric. In addition, the entanglement of fibers enhances the dimensional stability and overall strength of the fabric, while imparting some degree of flexibility to the fabric, so that it may be shaped or molded into any desired shape.

Additional embodiments of the disclosure may be associated with a method for providing access to the patient to minimize exposure of a healthcare worker to at least one of the patient's bodily fluids, bacteria, and viruses. According to an aspect, the method includes providing a protective equipment (e.g. similar to embodiments described herein, such as having a rotatable window and an extended member secured to the rotatable window and configured to prevent micro aerosolized particles from at least one of the patient's bodily fluids, bacteria, and viruses from traversing from an interior of the extended member to an exterior of the extended member). The rotatable window is disposed over the patient (e.g. when the patient is lying down on a surface). The method further includes positioning the extended member with respect to the patient to form the interior space, and applying negative pressure (e.g. reduced pressure, less than the surrounding atmospheric pressure of the exterior) within the interior space. According to an aspect, the method further includes accessing the patient, by a healthcare provider, while outside the interior space. During the accessing step, the healthcare provider has minimal exposure to at least one of the patient's bodily fluids, bacteria, and viruses. For example, the healthcare provider may access the patient in the interior space through one or more port. Some method embodiments may further include inserting one or more instruments into the interior space through the port. In some embodiments, the barrier of the port may help to protect the healthcare provider, even when one or more instrument is inserted through the port. Some embodiments may further include filtering the air, as described herein. Some embodiments may further include securing the extended member to the surface on which the patient is lying. In some embodiments, the interior space formed by the rotatable window and the extended member may be disposed around the patient's head/face. Some embodiments may further include the healthcare provider extending a hand through a sleeve in the port to access the patient (e.g. with the healthcare provider's hand then located in the interior space). These and other methods using the protective equipment are contemplated within the scope of this disclosure.

This disclosure, in various embodiments, configurations and aspects, includes components, methods, processes, systems, and/or apparatuses as depicted and described herein, including various embodiments, sub-combinations, and subsets thereof. This disclosure contemplates, in various embodiments, configurations and aspects, the actual or optional use or inclusion of, e.g., components or processes as may be well-known or understood in the art and consistent with this disclosure though not depicted and/or described herein.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

In this specification and the claims that follow, reference will be made to a number of terms that have the following meanings. The terms “a” (or “an”) and “the” refer to one or more of that entity, thereby including plural referents unless the context clearly dictates otherwise. As such, the terms “a” (or “an”), “one or more” and “at least one” can be used interchangeably herein. Furthermore, references to “one embodiment”, “some embodiments”, “an embodiment” and the like are not intended to be interpreted as excluding the existence of additional embodiments that also incorporate the recited features. Approximating language, as used herein throughout the specification and claims, may be applied to modify any quantitative representation that could permissibly vary without resulting in a change in the basic function to which it is related. Accordingly, a value modified by a term such as “about” or “substantially” is not to be limited to the precise value specified. In some instances, the approximating language may correspond to the precision of an instrument for measuring the value. Terms such as “first,” “second,” “upper,” “lower” etc. are used to identify one element from another, and unless otherwise specified are not meant to refer to a particular order or number of elements.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable, or suitable. For example, in some circumstances an event or capacity can be expected, while in other circumstances the event or capacity cannot occur—this distinction is captured by the terms “may” and “may be.”

As used in the claims, the word “comprises” and its grammatical variants logically also subtend and include phrases of varying and differing extent such as for example, but not limited thereto, “consisting essentially of” and “consisting of.” Where necessary, ranges have been supplied, and those ranges are inclusive of all sub-ranges therebetween. It is to be expected that the appended claims should cover variations in the ranges except where this disclosure makes clear the use of a particular range in certain embodiments.

The terms “determine”, “calculate” and “compute,” and variations thereof, as used herein, are used interchangeably and include any type of methodology, process, mathematical operation or technique.

This disclosure is presented for purposes of illustration and description. This disclosure is not limited to the form or forms disclosed herein. In the Detailed Description of this disclosure, for example, various features of some exemplary embodiments are grouped together to representatively describe those and other contemplated embodiments, configurations, and aspects, to the extent that including in this disclosure a description of every potential embodiment, variant, and combination of features is not feasible. Thus, the features of the disclosed embodiments, configurations, and aspects may be combined in alternate embodiments, configurations, and aspects not expressly discussed above. For example, the features recited in the following claims lie in less than all features of a single disclosed embodiment, configuration, or aspect. Thus, the following claims are hereby incorporated into this Detailed Description, with each claim standing on its own as a separate embodiment of this disclosure.

Advances in science and technology may provide variations that are not necessarily express in the terminology of this disclosure although the claims would not necessarily exclude these variations.

What is claimed is:

1. A protective equipment for use with a patient, the protective equipment comprising:
a rotatable window configured as a shield to be disposed between the patient and a healthcare worker to minimize exposure of the healthcare worker to at least one of the patient's bodily fluids, bacteria, and viruses; and
an extended member secured to a peripheral edge of the rotatable window, the extended member comprising
an inner surface configured to face the patient,
an outer surface opposite the inner surface, and
a port configured to provide access to the patient through the rotatable window or the extended member, wherein
the extended member is configured to prevent micro aerosolized particles from at least one of the patient's bodily fluids, bacteria, and viruses from traversing from an interior of the extended member to an exterior of the extended member, and
the port is configured to provide entrance by one or more instruments to provide access to the patient, while protecting the healthcare worker from at least one of the patient's bodily fluids, bacteria, and viruses.

2. The protective equipment of claim 1, wherein the rotatable window comprises:
a frame; and
a see-through body connected to the frame.

3. The protective equipment of claim 1, further comprising:
a support arm; and
a ball joint,
wherein the rotatable window is pivotably connected to the support arm via the ball joint, such that the rotatable window is vertically and laterally adjustable.

4. The protective equipment of claim 1, further comprising:
a barrier positioned in the port.

5. The protective equipment of claim 4, wherein
the barrier comprises a peripheral edge;
the port comprises a centralized location surrounded by the peripheral edge of the barrier; and
a plurality of deformable protrusions extends from the peripheral edge of the barrier towards the centralized location.

6. The protective equipment of claim 1, wherein the extended member comprises:
a first end secured to the peripheral edge of the rotatable window; and
a second end spaced apart from the first end and comprising a securing mechanism configured to retain the extended member in a spaced apart configuration from a body portion of the patient to define a space surrounding the body portion of the patient.

7. The protective equipment of claim 1, wherein the extended member comprises a material configured to trap aerosolized particles.

8. A protective equipment for use with a patient, the protective equipment comprising:
a rotatable window configured as a shield to be disposed between the patient and a healthcare worker to minimize exposure of the healthcare worker to at least one of the patient's bodily fluids, bacteria, and viruses;
an air flow system configured to pull air expelled by the patient away from the patient's face and filter the expelled air; and
an extended member secured to a peripheral edge of the rotatable window, the extended member comprising
an inner surface configured to face the patient,
an outer surface opposite the inner surface, and
a port positioned in the extended member extending between the inner surface and the outer surface, wherein
the extended member is configured to prevent micro aerosolized particles from at least one of the patient's bodily fluids, bacteria, and viruses from traversing from an interior of the extended member to an exterior of the extended member, and
the port provides entrance by one or more instruments through the extended member to provide access to the patient while protecting the healthcare worker from at least one of the patient's bodily fluids, bacteria and viruses.

9. The protective equipment of claim 8, wherein the air flow system comprises:
an air valve; and
an air outlet spaced apart from the air valve, wherein
the air valve is configured to receive and filter air from an environment external to the extended member, and
the air outlet is configured to pull the expelled air away from the patient's face and filter the expelled air.

10. The protective equipment of claim 9, further comprising:
a first filter,
wherein the first filter is positioned in the air valve and filters the air from the environment external to the extended member prior to the filtered air being received into an environment internal to the extended member.

11. The protective equipment of claim 9, wherein the air outlet is configured to pull the expelled air away from the patient's face towards an air filtration system.

12. The protective equipment of claim 11, wherein the air outlet comprises:
an air suction conduit configured to pull the expelled air away from the patient's face; and
a hose connected to the air suction conduit and having a first end and a second end, the first end of the hose being connected to the air suction conduit and the second end of the hose being connected to the air filtration system.

13. The protective equipment of claim 10, wherein the air outlet comprises an air suction conduit, and the air suction conduit comprises a second filter configured to filter the expelled air.

14. The protective equipment of claim 8, wherein the rotatable window comprises:
a frame; and
a see-through body connected to the frame.

15. The protective equipment of claim 12, wherein the rotatable window comprises:
a frame; and
a see-through body connected to the frame, wherein the air suction conduit extends through at least a portion of the frame.

16. A protective equipment for use with a patient, the protective equipment comprising:
a rotatable window configured as a shield to be disposed between the patient and a healthcare worker to minimize exposure of the healthcare worker to at least one of the patient's bodily fluids, bacteria, and viruses;
an extended member secured to a peripheral edge of the rotatable window, the extended member comprising
an inner surface configured to face the patient,
an outer surface opposite the inner surface, and
a port positioned in the extended member extending between the inner surface and the outer surface; and
a sleeve extending through the port, wherein
the extended member is configured to substantially inhibit or trap micro aerosolized particles from the least one of the patient's bodily fluids, bacteria, and viruses from traversing the extended member, and
the port provides entrance by the sleeve and an instrument through the extended member to provide access to the patient while protecting the healthcare worker from at least one of the patient's bodily fluids, bacteria, and viruses.

17. The protective equipment of claim 16, further comprising:
an air flow system configured to pull air expelled by the patient away from the patient's face and filter the expelled air.

18. The protective equipment of claim 17, wherein the air flow system comprises:
an air valve; and
an air outlet spaced apart from the air valve, wherein
the air valve is configured to receive and filter air from an environment external to the extended member, and
the air outlet is configured to pull the expelled air away from the patient's face towards an air filtration system to filter the expelled air.

19. The protective equipment of claim 18, wherein the air outlet comprises:
an air suction conduit having an intake section, wherein the intake section receives the patient's expelled air and the air suction conduit pulls the patient's expelled air away from the patient's face; and
a hose connected to the air suction conduit and having a first end and a second end, the first end of the hose being connected to the air suction conduit and the second end of the hose being connected to an air filtration system.

20. The protective equipment of claim 16, wherein the sleeve comprises:
a first end;
a second end;
a passage extending between the first end and the second end; and
a barrier, wherein
the barrier is positioned in the passage, and
the second end includes an elastic portion configured to securely wrap around the healthcare worker's wrist.
